(19) United States
(12) Patent Application Publication
(10) Pub. No.: US 2004/0063928 A1
(43) Pub. Date: Apr. 1, 2004

(54) Preparation of aliphatic acid ester of carbohydrate

(76) Inventor: In-Ho Jo, Cheungju-si (KR)

Correspondence Address: JORDAN AND HAMBURG LLP, 122 EAST 42ND STREET, SUITE 4000, NEW YORK, NY, 10168, US

(21) Appl. No.: 10/466,444

(22) PCT Filed: Jan. 17, 2002
(86) PCT No.: PCT/KR02/00076

(30) Foreign Application Priority Data
Jan. 18, 2001 (KR) 2001-0002787

(51) Int. Cl. C07H 13/02

(52) U.S. Cl. 536/119

(57) ABSTRACT

The present invention is a process for producing an aliphatic acid ester of carbohydrate comprising the steps of preparing an emulsion containing a salt of aliphatic acid and aqueous solution of carbohydrate or its derivative, obtaining a solid by removing water from said emulsion, and reacting said solid and an aliphatic acid ester.

### TECHNICAL FIELD

The present invention relates to a method for preparing fatty acid esters of carbohydrates or their derivatives which have a broad spectrum of applications in the food, pharmaceutical and cosmetic industries.

### BACKGROUND ART

Fatty acids esters of sugars, often just called sugar esters, are highly suitable for use as emulsifies in addition to being superior in terms of dispersibility.

Also, because sugar esters, which are decomposed to naturally occurring moieties, are non-toxic, tasteless, odorless, and non-irritating to the eyes and skin, they find useful applications particularly in the food, pharmaceutic, and cosmetic industries.

From the late 1950s to the early 1960s, much attention had been paid to the development of surfactants which can replace natural surfactants used in detergent compositions, drugs, cosmetics, baths and foods. Formerly, some salts of fatty acids were substituted with surfactants made of petroleum derivatives. However, because such surfactants were not so good in quality, much effort was made to take advantage of fatty acids obtainable from beef tallow and vegetable oils in producing useful surfactants. Of the surfactants developed, the most attractive are fatty acid esters of sugars.

For the last several decades, fatty acid esters of sugars have been extensively studied with great interest by many researchers, since they can be prepared from readily obtainable, natural materials such as sugars and beef tallow or vegetable oils.

Generally, sugars and animal or vegetable oils are not irritating to the skin and are physiologically acceptable, and are decomposed into non-toxic materials by microorganisms. With the physiological and environmental advantages, sugars and animal or vegetable oils have been extensively used as additives in cosmetics, drugs, foods, feedstuffs, and agricultural chemicals for freshening vegetables.

Typical examples of the sugars used in the preparation of fatty acid esters of sugars include sucrose, raffinose and glucose, with preference for sucrose. As for the fatty acids for the synthesis of fatty acid esters, they are typically exemplified by lauric acid, myristic acid, palmitic acid and stearic acid. Also, fatty acyl esters such as methyl palmitate, methyl stearate and methyl laurate are useful to prepare fatty acid esters of sugars through transesterification.

Largely, methods for preparing fatty acid esters of sugars can be classified into (1) a direct esterification method in which fatty acid chloride or fatty acid anhydrides are used, (2) an inter-esterification method in which fatty acid esters containing low alcohols are used, and (3) an enzymatic method in which esterification is conducted in the presence of an enzyme such as lipase.

The direct esterification method was, for the most part, applied for lab-scale production of fatty acid esters of sugars in the early stage of its development, and failed to enter a commercial stage owing to its economic disadvantages.

Also, the enzymatic method, even if still being attractive as a future method, has not yet succeeded in reaching a commercial stage.

In current industrial use is the method in which sugars are inter-esterified with methyl esters of fatty acids in the presence of a base catalyst. However, this method is disadvantageous in that the products cannot be used as food additives without complete removal of the anhydrous solvent N,N-dimethylformamide (DMF) or dimethyl sulfoxide (DMSO) from the reaction mixture. The inter-esterification method is disclosed in U.S. Pat. No. 2,893,990, yielded on Jul. 7, 1959. It is very difficult to isolate only sugar ester from the reaction mixture after the inter-esterification because the reaction mixture contains various materials, including the sugar ester, alkaline catalyst, anhydrous solvent, unreacted sugar, unreacted acyl ester and decomposed compounds.

A transparent emulsion method is well known to prepare fatty esters of sugars, especially fatty acid esters of sucrose. According to the transparent emulsion method, sucrose and fatty acyl ester are mixed, along with an emulsifier, in a solvent such as water to give a transparent emulsion which is then heated from 60° C. to 200° C. under an alkaline condition to afford fatty acid esters of sucrose, as disclosed in U.S. Pat. No. 3,644,333, yielded to Osipow on Feb. 22, 1972. However, the reaction mixture containing the product also contains many other materials such as unreacted starting materials, that is, sucrose and fatty acyl ester, decomposed materials from the starting materials, and the catalyst.

Although a lot of conditions necessary for the study on emulsifiers are suggested in patents and articles, it is needed to pay attention to the fact that reaction mixtures under conventional conditions cannot reach an emulsion state as described in example V of U.S. Pat. No. 3,644,333. The inventors of this reference patent confess a production yield of as low as 30˜35%. What is worse, it is difficult to isolate the product of interest from the reaction mixture which contains various by-products and reactants.

According to a typical example of the transparent emulsion method, the following reaction results are obtained:

| (a) Reactants | | |
|---|---|---|
| Sucrose | 80.4 | wt parts |
| Methyl stearate | 75 | wt parts |
| Sodium stearate | 12.3 | wt parts |
| Potassium stearate | 12.3 | wt parts |
| Potassium carbonate | 0.75 | wt parts |
| Water | 166.8 | wt parts |
| (b) Product | | |
| Sucrose monostearate | 40.5 | wt parts |

As seen in the above results, the conventional transparent emulsion method is not only poor in terms of production yield, but also leaves a significant amount of sodium stearate and alkaline fatty acid salts, thus failing to satisfy the requirement of the Food and Drug Administration of U.S.A. that a product should contain residues in an amount of 2% or less.

Another preparation method of fatty acid esters of sugars is found in U.S. Pat. No. 3,714,144, yielded to Feuge, which discloses that sodium, potassium and lithium salts of fatty acid are dissolved in a molten sugar and reacted with each other at 170-190° C. for 2-20 min. However, Feuge's method also suffers from the disadvantage of being very low in production yield and having difficulty in isolating the product of interest from the sugar and alkaline metal. In addition, Feuge's method, like Osipow's method, is inferior to an industrial process using a solvent in terms of product quality.

### DISCLOSURE OF THE INVENTION

Therefore, it is an object of the present invention to overcome the above problems encountered in prior arts and to provide a method for preparing a fatty acid ester of carbohydrate at high yield and with economical advantage.

Based on the present invention, the above object of the present invention could be accomplished by a provision of a method for preparing a fatty acid ester of a carbohydrate through ester-interchange between the carbohydrate or its derivative and fatty acid ester, comprising the steps of: emulsifying a solution of the carbohydrate or its derivative in water with a fatty acid salt to give an emulsion;

dehydrating the emulsion to leave a solid phase; transesterifying the solid phase with the fatty acid ester to produce the fatty acid ester of carbohydrate; and purifying the fatty acid ester of carbohydrate.

### BEST MODES FOR CARRYING OUT THE INVENTION

Preparation of Fatty Acid Esters of Sugars

In the present invention, the preparation of a fatty acid ester of a sugar starts with the dissolution of a carbohydrate or its derivative in water. The aqueous carbohydrate solution is emulsified by the addition of a salt of a fatty acid and the emulsion is dehydrated to give a solid phase which is then reacted with a fatty acid ester to produce a fatty acid ester of carbohydrate.

The carbohydrate or its derivative used as the starting material in the preparation of the emulsion is selected from the group consisting of monosaccharides, disaccharides, polysaccharides, their derivatives, and mixtures thereof. Preferred are sucrose, glucose, fructose, galactose, 6-deoxygalactose, xylose, ribose, arabinose, lactose, maltose, palatinose, melibiose, talose, 2-deoxyglucose, mannose, 6-deoxymannose, sophorose, raffinose, and cellobiose.

Suitable for use in the preparation of the emulsion is a fatty acid salt selected from the group consisting of alkali metal salts (e.g., potassium and sodium salts) and alkaline earth metal salts (e.g., calcium salt) of fatty acids containing 8-22 carbon atoms, and mixtures thereof.

To facilitate the emulsification of the aqueous carbohydrate solution, an emulsification promoter may be used. Examples of the emulsification promoter include hydrogen, oxygen, nitrogen, hydrogen peroxide, nitric oxide, nitrogen dioxide, potassium hydroxide, sodium hydroxide, lithium hydroxide, potassium peroxide, sodium peroxide, lithium peroxide, potassium carbonate, sodium carbonate, lithium carbonate, potassium bicarbonate, sodium bicarbonate, lithium bicarbonate, potassium methylate, sodium methylate, lithium methylate, potassium ethylate, sodium ethylate, lithium ethylate, potassium propylate, sodium propylate, potassium butylate, sodium butylate, and lithium butylate. Also, potassium hydroxide, sodium hydroxide, lithium hydroxide, potassium peroxide, sodium peroxide, lithium peroxide, potassium carbonate, sodium carbonate, lithium carbonate, potassium bicarbonate, sodium bicarbonate, lithium bicarbonate, potassium methylate, sodium methylate, lithium methylate, potassium ethylate, sodium ethylate, lithium ethylate, potassium propylate, sodium propylate, potassium butylate, sodium butylate, and lithium butylate can function as catalysts for ester interchange, later.

In accordance with the present invention, the reaction of carbohydrates or their derivatives with fatty acid esters, that is, transesterification for producing fatty acid esters of carbohydrates, is conducted not under an emulsion condition, but in a homogeneous solid of fine particles obtained by completely dehydrating the emulsion.

To find optimal conditions for the transesterification, the reaction mixture and product were analyzed with the aid of a Fourier transform infrared (FT-IR) spectroscope and a thin layer chromatography (TLC) analyzer. The results show that the transesterification is effectively performed at 140-175° C. after a catalyst is added to a reaction mixture heated to 130-140° C. Also, the transesterification may be conducted at atmospheric pressure or at a reduced pressure of 0-60 mmHg, with preference for the reduced pressure. However, it should be understood that the esterification conditions are not construed to limit the present invention.

The transesterification reaction time and temperature are dependent on the length of the carbon chain of the fatty acid used. When employing a longer carbon chain of the fatty acid, the transesterification can be completed at a lower temperature within a shorter time. For example, when the carbon chain length of the fatty acid is 16 or more, the reaction is preferably conducted for 2-4 hours at 140-160° C. When the carbon chain length is less than 16, the reaction time is extended to 6-8 hours while the reaction temperature is increased to 150-175° C.

Suitable for use in the transesterification of the present invention are esters of C₆-C₂₂ fatty acids. Particularly suitable are esters of C₆-C22 fatty acids which are prepared by esterifying one or more C₆-C₂₂ fatty acids with one or more C₁-C₅ mono- or poly-alcohols. Preferable examples of the C₁-C₅ mono- or poly-alcohols include methanol, ethanol, propanol, butanol, ethylene glycol, propylene glycol, butylene glycol, glycerol, sorbitol and pentaerythritol.

Particularly advantageous in producing fatty acid esters of sugars with high purity are fatty acid esters which have low-boiling point alcohol groups such as methanol, ethanol and propanol.

As above-mentioned, the transesterification between fatty acid esters and alcohols may be achieved in the presence of a catalyst. A suitable one is selected from the group consisting of potassium hydroxide, sodium hydroxide, lithium hydroxide, potassium peroxide, sodium peroxide, lithium peroxide, potassium carbonate, sodium carbonate, lithium carbonate, potassium bicarbonate, sodium bicarbonate, lithium bicarbonate, potassium methylate, sodium methylate, lithium methylate, potassium ethylate, sodium ethylate, lithium ethylate, potassium propylate, sodium propylate, potassium butylate, sodium butylate, lithium butylate, and mixtures thereof. More preferable is a potassium salt such as potassium carbonate or potassium hydroxide.

Purification of Fatty Acid Esters of Carbohydrates

After the transesterification between carbohydrates or their derivatives and fatty acid esters, an additional process is needed to isolate the fatty acid esters of carbohydrates from the reaction mixture. In this regard, conventional purification processes may be used, but the present invention adopts the following processes for higher efficiency.

(i) To the reaction mixture obtained after the transesterification, water and an organic solvent with a boiling point lower than that of water are added, followed by stirring it to give an emulsion. The organic solvent suitable for use in the preparation of the emulsion is selected from the group consisting of aliphatic alcohols containing 1-4 carbon atoms, ketones containing 3-6 carbon atoms, ethers containing 3-6 carbon atoms, esters containing 3-5 carbon atoms, halogen compounds containing 1-4 carbon atoms, and mixtures thereof.

(ii) The addition of an aqueous solution of a neutral salt divides the emulsion into two phases: an organic phase containing desired fatty acid esters of carbohydrates, fatty acid salts, and unreacted fatty acid esters; and an aqueous phase containing unreacted carbohydrates and their derivatives. The two phases can be readily separated by a simple physical operation. The neutral salt is preferably selected from the group consisting of sodium chloride, potassium chloride, lithium chloride, sodium bromide, potassium bromide, lithium bromide, sodium iodide, potassium iodide, lithium iodide, Glauber's salt, and mixtures thereof.

(iii) After being separated, the organic phase is mixed with a low-boiling point organic solvent to precipitate salts of fatty acids owing to their low solubility. The organic solvent is selected from the group consisting of C₄-Cs ethers, C₃-C₆ ketones, C₃-C₅ esters, and mixtures thereof. Filtration results in the separation of the liquid phase containing fatty acid esters of carbohydrates and unreacted fatty acid esters from the solid phase of salts of fatty acids.

(iv) Addition of water to the filtrate forms an aqueous phase containing fatty acid esters of carbohydrates with high HLB values, and an organic phase containing fatty acid esters of carbohydrates with low HLB values and unreacted fatty acid esters. These two phases can be physically separated with ease.

(v) The aqueous phase containing fatty acid esters of carbohydrates with high HLB values and the organic phase containing fatty acid esters of carbohydrates with low HLB values and unreacted fatty acid esters are subjected to the following respective purification to reclaim fatty acid esters of carbohydrates.

From the aqueous phase, fatty acid esters of carbohydrates with high HLB values are isolated as follows:

(A-1) The aqueous phase is added with a low-boiling point organic solvent and a neutral salt-saturated aqueous solution to concentrate the fatty acid esters of carbohydrates with high HLB values in the organic phase rather than in the resulting aqueous phase. The two phases are physically separated with ease. The organic solvent suitable for this purpose is selected from the group consisting of ketones containing 3-6 carbon atoms, halogen compounds containing 1-4 carbon atoms, esters containing 3-5 carbon atoms, and mixtures thereof.

(A-2) The organic solvent is removed from the organic phase containing fatty acid esters of carbohydrates by vacuum evaporation, and the residue is added with a low-boiling point organic solvent to form precipitates which are then obtained by filtration. The low-boiling point organic solvent is preferably selected from the group consisting of C₁-C₄ aliphatic alcohols, C₃-C₆ ketones, C₃-C₅ esters, and mixtures thereof.

(A-3) The precipitate is washed with a low-boiling organic solvent and dried to afford fatty acid esters of carbohydrates (when using sucrose, the monoester content amounts to about 60-70%). The washing organic solvent is preferably selected from the group consisting of C₃-C₆ ketones, C₄-C₈ ethers, C₃-C₅ esters, and mixtures thereof.

(A-4) From the filtrate thus obtained in (A-2), the organic solvent is removed, and the residue (a pale yellow soft material when using sucrose) is added with a low-boiling solvent to precipitate fatty acid esters of carbohydrates. The fatty acid esters of sucrose contains monoesters in an amount of about 80-95%. A suitable low-boiling solvent in this step is selected from the group consisting of C₁-C₄ aliphatic alcohols, C₄-C₈ ethers, C₃-C₅ esters, and mixtures thereof.

The organic phase containing fatty acid esters of carbohydrates with low HLB values and unreacted fatty acid esters is rendered to undergo the following isolation processes.

(B-1) By vacuum distillation, the organic phase containing fatty acid esters of carbohydrates with low HLB values and unreacted fatty acid esters is concentrated to a slurry state to which a low-boiling point organic solvent is subsequently added, so as to form a precipitate which is separated from the liquid phase by filtration. The organic solvent is preferably selected from the group consisting of halogen compounds containing 1-4 carbon atoms, ketones containing 3-6 carbon atoms, esters containing 3-5 carbon atoms, and mixtures thereof.

(B-2) The precipitate is washed with an organic solvent and dried to produce fatty acid esters of carbohydrates (the monoester content amounts to 0-10% when using sucrose).

(B-3) Removal of the organic solvent from the filtrate thus obtained in (B-1) leaves a soft material. To this residue is added an organic solvent with a low boiling point, so as to form a precipitate. The organic solvent suitable for the precipitation is preferably selected from the group consisting of C₃-C₆ ketones, C₃-Cs esters, C4-Cs ethers, and mixtures thereof. Separation of the precipitate from the liquid phase resorts to filtration. In the liquid phase, unreacted fatty acid esters remain dissolved, which can be separated by vacuum distillation. Washing the precipitate with an organic solvent and drying it gives fatty acid esters of carbohydrates (the monoester content amounts to about 20-40% when using sucrose). In all of the purification steps, organic solvents selected from group consisting of aliphatic alcohols containing 1-4 carbon atoms, ketones containing 3-6 carbon atoms, ethers containing 4-8 carbon atoms, esters containing 3-5 carbon atoms, halogen compounds containing 1-4 carbon atoms, and mixtures thereof may be used, but different organic solvents are preferably selected for consecutive steps. Useful materials obtained from each purification step, including fatty acid salts, unreacted fatty acid esters, organic solvents, etc., may be reused in subsequent preparation and purification processes for fatty acid esters of carbohydrates.

The purification processes of the present invention, although described in relation to the preparation of fatty acid esters of carbohydrates, may be very usefully applied for the isolation of esters of interest produced by ester interchange.

Having generally described this invention, a further understanding can be obtained by reference to certain specific examples which are provided herein for purposes of illustration only and are not intended to be limiting unless otherwise specified.

### EXAMPLE 1

In 100 mL of water was dissolved 150.0 g of sucrose, and the aqueous solution was stirred, along with 50 g of sodium stearate, at 60° C. for 1 hour to give an emulsion.

Complete removal of the water from the emulsion resulted in a solid. This was heated, along with 70.0 g of methyl stearate, in a reactor. When the temperature was increased to 140° C., 5.0 g of potassium carbonate was added into the reactor, followed by increasing the temperature under a pressure of 20-60 mmHg to 160° C., under which conditions the reaction mixture was stirred for 4 hours.

After isolation and purification of the product from the reaction mixture, an analysis showed that as little as 6% of the methyl stearate added was converted into sucrose stearate by ester interchange. Through thin layer chromatography, the sucrose stearate was found to comprise mono-, di- and tri-esters in a composition ratio shown in Table 1, below.

### EXAMPLE 2

To a solution of 150 g of sucrose in 100 mL of water were added 50.0 g of sodium stearate and 5.0 mL of a 30% aqueous solution of hydrogen peroxide, followed by stirring the resulting solution at 60° C. for 1 hour. Complete dehydration of the solution gave a solid to which 70.0 g of methyl stearate was then added. In a reactor, the mixture was heated to 140° C. before the addition of 5.0 g of potassium carbonate, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of as high as 87%. Through thin layer chromatography, the sucrose stearate was found to comprise mono-, di- and tri-esters in a composition ratio shown in Table 1, below.

### EXAMPLE 3

To a solution of 150 g of sucrose in 100 mL of water were added 50.0 g of sodium stearate and 5.0 g of potassium carbonate, followed by stirring the resulting solution at 60° C. for 1 hour. Complete dehydration of the solution gave a solid to which 70.0 g of methyl stearate was then added. In a reactor, the mixture was heated at a pressure of 20-60 mmHg to 160° C. under which conditions stirring was conducted for 4 hours.

After isolation and purification of the product from the reaction mixture, analysis showed that the methyl stearate added was converted into sucrose stearate by ester interchange at a conversion rate of 11%. Through thin layer chromatography, the sucrose stearate was found to comprise mono-, di- and tri-esters in a composition ratio shown in Table 1, below.

### EXAMPLE 4

To a solution of 150 g of sucrose in 100 mL of water were added 50.0 g of sodium stearate and 2.3 g of potassium carbonate, followed by stirring the resulting solution at 60° C. for 1 hour. Complete dehydration of the solution gave a solid to which 70.0 g of methyl stearate was then added. In a reactor, the mixture was heated to 140° C. before the addition of 2.7 g of potassium carbonate, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of 15%. Through thin layer chromatography, the sucrose stearate was found to comprise mono-, di- and tri-esters in a composition ratio shown in Table 1, below.

### EXAMPLE 5

To a solution of 150 g of sucrose in 100 mL of water were added 50.0 g of sodium stearate and 0.3 g of potassium carbonate, followed by stirring the resulting solution at 60° C. for 1 hour. Complete dehydration of the solution gave a solid to which 70.0 g of methyl stearate was then added. In a reactor, the mixture was heated to 140° C. before the addition of 4.7 g of potassium carbonate, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of 27%. Through thin layer chromatography, the sucrose stearate was found to comprise mono-, di- and tri-esters in a composition ratio shown in Table 1, below.

### EXAMPLE 6

To a solution of 150 g of sucrose in 100 mL of water were added 50.0 g of sodium stearate and 0.1 g of potassium hydroxide, followed by stirring the resulting solution at 60° C. for 1 hour. Complete dehydration of the solution gave a solid to which 70.0 g of methyl stearate was then added. In a reactor, the mixture was heated to 140° C. before the addition of 1.9 g of potassium hydroxide, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of 15%. Through thin layer chromatography, the sucrose stearate was found to comprise mono-, di- and tri-esters in a composition ratio shown in Table 1, below.

### EXAMPLE 7

To a solution of 150 g of sucrose in 100 mL of water were added 50.0 g of sodium stearate and 0.2 g of potassium bicarbonate, followed by stirring the resulting solution at 60° C. for 1 hour. Complete dehydration of the solution gave a solid to which 70.0 g of methyl stearate was then added. In a reactor, the mixture was heated to 140° C. before the addition of 4.7 g of potassium carbonate, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of 20%. Through thin layer chromatography, the sucrose stearate was found to comprise mono-, di- and tri-esters in a composition ratio shown in Table 1, below.

### EXAMPLE 8

To a solution of 150 g of sucrose in 100 mL of water were added 50.0 g of sodium stearate and 0.2 g of sodium peroxide, followed by stirring the resulting solution at 60° C. for 1 hour. Complete dehydration of the solution gave a solid to which 70.0 g of methyl stearate was then added. In a reactor, the mixture was heated to 140° C. before the addition of 4.7 g of potassium carbonate, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of 17%. Through thin layer chromatography, the sucrose stearate was found to comprise mono-, di- and tri-esters in a composition ratio shown in Table 1, below.

### EXAMPLE 9

To a solution of 150 g of sucrose in 100 mL of water were added 50.0 g of sodium stearate and 0.1 g of sodium methylate, followed by stirring the resulting solution at 60° C. for 1 hour. Complete dehydration of the solution gave a solid to which 70.0 g of methyl stearate was then added. In a reactor, the mixture was heated to 140° C. before the addition of 4.7 g of potassium carbonate, and the temperature was further increased at a pressure of 20-60 mmHg to 160° C. at which stirring was then conducted for 4 hours.

After isolation and purification of the product from the reaction mixture, analysis showed that the methyl stearate added was converted into sucrose stearate by ester interchange at a conversion rate of 10%. Through thin layer chromatography, the sucrose stearate was found to comprise mono-, di- and tri-esters in a composition ratio shown in Table 1, below.

### EXAMPLE 10

To a solution of 150 g of sucrose in 100 mL of water were added 45.3 g of sodium palmiate and 5.0 mL of hydrogen peroxide, followed by stirring the resulting solution at 60° C. for 1 hour. Complete dehydration of the solution gave a solid to which 63.7 g of methyl palmitate was then added. In a reactor, the mixture was heated to 140° C. before the addition of 5.0 g of potassium carbonate, and the temperature was further increased at a pressure of 20-60 mmHg to 160° C. at which stirring was then conducted for 4 hours.

After isolation and purification of the product from the reaction mixture, analysis showed that the methyl palmitate added was converted into sucrose palmitate by ester interchange at a conversion rate of 81%. Through thin layer chromatography, the sucrose palmitate was found to comprise mono-, di- and tri-esters in a composition ratio shown in Table 1, below.

### EXAMPLE 11

To a solution of 150 g of sucrose in 100 mL of water were added 36.3 g of sodium laurate and 5.0 mL of hydrogen peroxide, followed by stirring the resulting solution at 60° C. for 1 hour. Complete dehydration of the solution gave a solid to which 50.3 g of methyl laurate was then added. In a reactor, the mixture was heated to 140° C. before the addition of 5.0 g of potassium carbonate, and the temperature was further increased at a pressure of 20-60 mmHg to 170° C. at which stirring was then conducted for 6 hours.

After isolation and purification of the product from the reaction mixture, analysis showed that the methyl laurate added was converted into sucrose laurate by ester interchange at a conversion rate of 83%. Through thin layer chromatography, the sucrose laurate was found to comprise mono-, di- and tri-esters in a composition ratio shown in Table 1, below.

### EXAMPLE 12

To a solution of 150 g of sucrose in 100 mL of water were added 50.0 g of sodium stearate and 5.0 mL of hydrogen peroxide, followed by stirring the resulting solution at 60° C. for 1 hour. Complete dehydration of the solution gave a solid to which 50.3 g of methyl laurate was then added. In a reactor, the mixture was heated to 140° C. before the addition of 5.0 g of potassium carbonate, and the temperature was further increased at a pressure of 20-60 mmHg to 170° C. at which stirring was then conducted for 6 hours.

After isolation and purification of the product from the reaction mixture, analysis showed that the methyl laurate added was converted into sucrose laurate by ester interchange at a conversion rate of 85%. Through thin layer chromatography, the sucrose laurate was found to comprise mono-, di- and tri-esters in a composition ratio shown in Table 1, below.

### EXAMPLE 13

To a solution of 150 g of sucrose in 100 mL of water were added 50.0 g of sodium stearate and 5.0 mL of hydrogen peroxide, followed by stirring the resulting solution at 60° C. for 1 hour. Complete dehydration of the solution gave a solid to which 69.7 g of methyl oleate was then added. In a reactor, the mixture was heated to 140° C. before the addition of 5.0 g of potassium carbonate, and the temperature was further increased at a pressure of 20-60 mmHg to 160° C. at which stirring was then conducted for 4 hours.

After isolation and purification of the product from the reaction mixture, analysis showed that the methyl oleate added was converted into sucrose oleate by ester interchange at a conversion rate of 82%. Through thin layer chromatography, the sucrose oleate was found to comprise mono-, di- and tri-esters in a composition ratio shown in Table 1, below.

### EXAMPLE 14

To a solution of 150 g of sucrose in 100 mL of water were added 50.0 g of sodium stearate and 5.0 mL of hydrogen peroxide, followed by stirring the resulting solution at 60° C. for 1 hour. Complete dehydration of the solution gave a solid to which 83.3 g of methyl behenate was then added. In a reactor, the mixture was heated to 140° C. before the addition of 5.0 g of potassium carbonate, and the temperature was further increased at a pressure of 20-60 mmHg to 160° C. at which stirring was then conducted for 4 hours.

After isolation and purification of the product from the reaction mixture, analysis showed that the methyl behenate added was converted into sucrose behenate by ester interchange at a conversion rate of 75%. Through thin layer chromatography, the sucrose behenate was found to comprise mono-, di- and tri-esters in a composition ratio shown in Table 1, below.

### EXAMPLE 15

To a solution of 150 g of sucrose in 100 mL of water were added 50.0 g of sodium stearate and 5.0 mL of hydrogen peroxide, followed by stirring the resulting solution at 60° C. for 1 hour. Complete dehydration of the solution gave a solid to which 82.7 g of methyl erucate was then added. In a reactor, the mixture was heated to 140° C. before the addition of 5.0 g of potassium carbonate, and the temperature was further increased at a pressure of 20-60 mmHg to 160° C. at which stirring was then conducted for 4 hours.

After isolation and purification of the product from the reaction mixture, analysis showed that the methyl erucate added was converted into sucrose erucate by ester interchange at a conversion rate of 65%. Through thin layer chromatography, the sucrose erucate was found to comprise mono-, di- and tri-esters in a composition ratio shown in Table 1, below.

TABLE 1

| | | Con- | Composition of | | |
|---|---|---|---|---|---|
| | | version | Sucrose Ester (wt %) | | |
| Exam. | Kind of | Rate | | | Triester |
| No. | Sucrose Ester | (%) | Monoester | Diester | & poly ester |
| 1 | Sucrose stearate | 6 | 48 | 32 | 20 |
| 2 | Sucrose stearate | 87 | 83 | 12 | 5 |
| 3 | Sucrose stearate | 11 | 23 | 57 | 20 |
| 4 | Sucrose stearate | 15 | 31 | 48 | 21 |
| 5 | Sucrose stearate | 27 | 52 | 31 | 17 |
| 6 | Sucrose stearate | 15 | 45 | 33 | 22 |
| 7 | Sucrose stearate | 20 | 50 | 35 | 15 |
| 8 | Sucrose stearate | 17 | 47 | 30 | 23 |
| 9 | Sucrose stearate | 10 | 40 | 37 | 23 |
| 10 | Sucrose palmitate | 81 | 80 | 15 | 5 |
| 11 | Sucrose laurate | 83 | 75 | 17 | 8 |
| 12 | Sucrose laurate | 85 | 78 | 15 | 7 |
| 13 | Sucrose oleate | 82 | 81 | 12 | 7 |
| 14 | Sucrose behenate | 75 | 85 | 10 | 5 |
| 15 | Sucrose erucate | 65 | 77 | 19 | 4 |

### EXAMPLE 16

The reaction mixture obtained after the ester interchange in Example 2 was cooled to 30° C. and then stirred, together with 100 mL of water, 150 mL of chloroform and 50 mL of ethanol, in a mixer, to give an emulsion which was subsequently divided into an organic phase [I] and an aqueous phase [II] by the addition of 7 mL of an aqueous sodium chloride-saturated solution.

After separation, the organic phase [I] was added with 50 mL of acetone to form a precipitate, identified as sodium stearate, which was obtained by filtration.

Addition of 100 mL of water separated the resulting filtrate into an aqueous phase [III] and an organic phase [IV].

To the aqueous phase [III], 50 mL of chloroform and 7 mL of a sodium chloride-saturated aqueous solution were added, to form an organic phase [V] and an aqueous phase [VI].

Chloroform was removed from the organic phase [V] by vacuum distillation, and the residue was added with 50 mL of acetone to form a precipitate which was then obtained by filtration while leaving a filtrate [VII].

The precipitate was washed with 80 mL of ethyl acetate and dried to afford 87.0 g of sucrose stearate which contained monostearate in an amount of 60-70 wt %.

By vacuum distillation, the filtrate [VII] was removed of acetone. To the residue thus obtained as a yellowish soft material, 50 mL of ethyl acetate was added, to precipitate a solid material which was obtained by filtration. Then, the precipitate was washed with 50 mL of ethyl acetate and dried to give 23.2 g of sucrose stearate whose monostearate content amounted to as high as 85-95 wt %.

Separately, the organic phase [IV] was distilled in vacuo to obtain a slurry which was mixed with 30 mL of acetone, to form a precipitate. This was filtered while leaving a filtrate [VIII]. The precipitate was washed with 30 mL of ethyl acetate and dried to afford 14.7 g of sucrose stearate with a monoester content of 0-10 wt %.

Then, the filtrate [VII] was removed of acetone by vacuum distillation, and the soft residue thus obtained was mixed with 20 niL of ethyl acetate to form a precipitate which was subsequently filtered, washed with 30 ML of ethyl acetate and dried to yield 7.2 g of sucrose stearate in which a monoester was contained in an amount of 20-40 wt %.

From the filtrate thus formed, discriminated by an I.D. No. [IX], ethyl acetate and unreacted methyl stearate were isolated by vacuum distillation.

### INDUSTRIAL APPLICABILITY

The sucrose stearate product obtained in one purification step is different in monostearate content from that obtained in another step, and the sucrose stearate products can be used for different purposes. In addition, the useful materials isolated from each purification step, including sodium stearate, chloroform, ethanol, acetone, ethyl acetate and methyl stearate, can be reused.

As described hereinbefore, a solid phase obtained by almost completely removing the solvent from an emulsion of carbohydrates or their derivatives can be trans-esterified with fatty acid esters to produce fatty acid esters of carbohydrates at high yield, as well as making the purification of the products easy.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A method for preparing a fatty acid ester of a carbohydrate through ester-interchange between the carbohydrate or its derivative and fatty acid ester, comprising the steps of:
emulsifying a solution of the carbohydrate or its derivative in water with a fatty acid salt to give an emulsion;
dehydrating the emulsion to leave a solid phase;
transesterifying the solid phase with the fatty acid ester to produce the fatty acid ester of the carbohydrate; and
purifying the fatty acid ester of the carbohydrate.

2. The method as set forth in claim 1, wherein the emulsifying step is carried out by use of an emulsification promoter.

3. The method as set forth in claim 1, wherein the transesterfying step is carried out in the presence of a transesterification catalyst.

4. The method as set forth in claim 1, wherein the emulsifying step is carried out in the presence of a transesterification catalyst.

5. The method as set forth in claim 1, wherein the carbohydrate or its derivative is selected from the group consisting of monosaccharides, disaccharides, polysaccharides, their derivatives, and mixtures thereof.

6. The method as set forth in claim 5, wherein the carbohydrate is a sugar.

7. The method as set forth in claim 6, wherein the sugar is selected from the group consisting of sucrose, glucose, fructose, galactose, 6-deoxygalactose, xylose, ribose, arabinose, lactose, maltose, palatinose, melibiose, talose, 2-deoxyglucose, mannose, 6-deoxymannose, sophorose, raffinose, and cellobiose.

8. The method as set forth in claim 1, wherein the fatty acid salt is selected from the group consisting of alkali metal salts and alkaline earth metal salts of C₈-C₂₂ fatty acids, and mixtures thereof.

9. The method as set forth in claim 8, wherein the fatty acid salt is a potassium salt, a sodium salt, or a calcium salt of C₈-C₂₂ fatty acids.

10. The method as set forth in claim 2, wherein the emulsification promoter is selected from the group consisting of hydrogen, oxygen, nitrogen, hydrogen peroxide, nitric oxide, nitrogen dioxide, potassium hydroxide, sodium hydroxide, lithium hydroxide, potassium peroxide, sodium peroxide, lithium peroxide, potassium carbonate, sodium carbonate, lithium carbonate, potassium bicarbonate, sodium bicarbonate, lithium bicarbonate, potassium methylate, sodium methylate, lithium methylate, potassium ethylate, sodium ethylate, lithium ethylate, potassium propylate, sodium propylate, potassium butylate, sodium butylate, lithium butylate, and mixtures thereof.

11. The method as set forth in claim 1, wherein the emulsifying step is carried out at 40-60° C. for 1-2 hours with stirring.

12. The method as set forth in claim 1, wherein the fatty acid ester is an ester of C₆-C₂₂ fatty acids.

13. The method as set forth in claim 12, wherein the fatty acid ester is a product resulting from the esterification between a C₆-C₂₂ fatty acid or a mixture of C₆-C₂₂ fatty acids and an alcohol selected from the group consisting of C₁-C₅ mono- and poly-alcohols and mixtures thereof.

14. The method as set forth in claim 13, wherein the alcohol is methanol, ethanol, propanol, butanol, ethylene glycol, propylene glycol, butylene glycol, glycerol, sorbitol, or pentaerythritol.

15. The method as set forth in claim 14, wherein the alcohol is methanol, ethanol or propanol.

16. The method as set forth in claim 3 or 4, wherein the transesterification catalyst is selected from the group consisting of potassium hydroxide, sodium hydroxide, lithium hydroxide, potassium peroxide, sodium peroxide, lithium peroxide, potassium carbonate, sodium carbonate, lithium carbonate, potassium bicarbonate, sodium bicarbonate, lithium bicarbonate, potassium methylate, sodium methylate, lithium methylate, potassium ethylate, sodium ethylate, lithium ethylate, potassium propylate, sodium propylate, potassium butylate, sodium butylate, lithium butylate, and mixtures thereof.

17. The method as set forth in claim 16, wherein the transesterification catalyst is potassium carbonate or potassium hydroxide.

18. The method as set forth in claim 1, wherein the fatty acid ester is an ester of C₁₆-C₂₂ fatty acids and the transesterifying step is carried out at 140-165° C. for 2-4 hours.

19. The method as set forth in claim 1, wherein the fatty acid ester is an ester of C₆-C₁₅ fatty acids and the transesterifying step is carried out at 150-175° C. for 6-8 hours.

20. The method as set forth in claim 1, wherein the transesterifying step is carried out at a pressure of 0-60 mmHg.

21. The method as set forth in claim 1, wherein the purifying step comprises:
mixing the reaction mixture obtained after the transesterifying step with water or an organic solvent, with stirring, to give an emulsion, the organic solvent being lower in boiling point than water;
adding an aqueous solution of a neutral salt to the emulsion to form an organic layer containing fatty acid esters of carbohydrates, salts of fatty acids, and unreacted fatty acid esters, and an aqueous layer containing unreacted carbohydrates or their derivatives;
precipitating the fatty acid salts in the organic layer by taking advantage of the low solubility of the salts in a low-boiling point organic solvent, and separating the precipitate from the liquid;
dividing the liquid into an aqueous phase and an organic phase by the addition of water, said aqueous phase containing fatty acid esters of carbohydrates with high HLB values, said organic phase containing fatty acid esters of carbohydrates with low HLB values, and unreacted fatty acid esters; and
isolating fatty acid esters of carbohydrates with high and low HLB values from the aqueous and the organic phase, respectively.

22. The method as set forth in claim 21, wherein the isolation of the fatty acid esters of carbohydrates with high HLB values from the aqueous phase is carried out by a process comprising the steps of:
adding to the aqueous phase a low-boiling point organic solvent and an aqueous solution saturated with a neutral salt to form an organic layer and an aqueous layer;
distilling the organic layer in vacuo to remove the organic solvent, followed by mixing the residue with a low-boiling point organic solvent to afford the fatty acid esters of carbohydrates as a precipitate, and separating the precipitate from the liquid by filtration; and
removing the organic solvent from the remaining liquid, followed by mixing the residue with a low-boiling point organic solvent to afford the fatty acid esters of carbohydrates as a precipitate, and filtering the precipitate.

23. The method as set forth in claim 21, wherein the isolation of the fatty acid esters of carbohydrates with low HLB values from the organic phase is carried out by a process comprising the steps of:
distilling the organic phase in vacuo to give a slurry, followed by adding a low-boiling point organic solvent to the slurry to form a precipitate and separating the precipitate from the liquid through filtration;
Reclaiming the precipitate as a fatty acid ester of carbohydrate by washing with an organic solvent and drying; and
removing the organic solvent from the liquid, followed by mixing the residue with a low-boiling point organic solvent to afford the fatty acid esters of carbohydrates as a precipitate and filtering the precipitate.

24. The method as set forth in one of claims 21 to 23, wherein the organic solvents are selected from the group consisting of aliphatic alcohols containing 1-4 carbon atoms, ketones containing 3-6 carbon atoms, esters containing 3-5 carbon atoms, halogen compounds containing 1-4 carbon atoms, and mixtures thereof.

25. The method as set forth in claim 24, wherein the organic solvents used in consecutive steps are different from each other.

26. The method as set forth in one of claims 21 to 23, wherein the neutral salt is selected from the group consisting of sodium chloride, potassium chloride, lithium chloride, sodium bromide, potassium bromide, lithium bromide, sodium iodide, potassium iodide, lithium iodide, Glauber's salt, and mixtures thereof.

27. A method for purifying fatty acid esters of carbohydrates or their derivatives from the reaction mixtures of the transesterification between carbohydrates or their derivatives and fatty acid esters, comprising the steps of:
mixing the reaction mixtures with water and an organic solvent lower in boiling point than water, with stirring to form an emulsion;
adding an aqueous solution of a neutral salt to the emulsion to form an organic layer containing fatty acid esters of carbohydrates, salts of fatty acids, and unreacted fatty acid esters, and an aqueous layer containing unreacted carbohydrates or their derivatives;
precipitating the fatty acid salts in the organic layer by taking advantage of the low solubility of the salts in a low-boiling point organic solvent, and separating the precipitate from the liquid;
dividing the liquid into an aqueous phase and an organic phase by the addition of water, said aqueous phase containing fatty acid esters of carbohydrates with high HLB values, said organic phase containing fatty acid esters of carbohydrates with low HLB values, and unreacted fatty acid esters; and
isolating fatty acid esters of carbohydrates with high and low HLB values from the aqueous and the organic phase, respectively.
